(19) United States
(12) Patent Application Publication
(10) Pub. No.: US 2025/0194933 A1
(43) Pub. Date: Jun. 19, 2025

(54) INTRA ORAL SCANNER AND COMPUTER IMPLEMENTED METHOD FOR UPDATING A DIGITAL 3D SCAN

(71) Applicant: 3SHAPE A/S, Copenhagen K (DK)

(72) Inventor: Asger Vejen HOEDT, Copenhagen K (DK)

(73) Assignee: 3SHAPE A/S, Copenhagen K (DK)

(21) Appl. No.: 18/847,941

(22) PCT Filed: Mar. 15, 2023
(86) PCT No.: PCT/EP2023/056603

§ 371 Date: Sep. 17, 2024

(30) Foreign Application Priority Data
Mar. 17, 2022 (EP) 22162644.3

(51) Int. Cl. A61B 5/00; G06T 7/00; G06T 19/20

(52) U.S. Cl. CPC A61B 5/0088; G06T 7/0016; G06T 19/20; G06T 2207/30036

(57) ABSTRACT

According to an embodiment, a computer implemented method and a scanner system are disclosed. The computer implemented method for updating a current digital 3D scan representing the surface of a physical object with an at least one new 3D scan, where the updating of the current digital 3D scan provides an updated digital 3D scan representation of the surface of the physical object is disclosed. The computer implemented method may comprise obtaining the current digital 3D scan, obtaining the at least one new digital 3D scan, determining an inconsistent digital 3D scan, where at least a part of the new digital 3D scan does not overlap with at least a part of the current digital 3D scan, and creating the updated digital 3D scan, which represent the physical object by applying the inconsistent digital 3D scan to the current digital 3D scan.

### FIELD

The disclosure relates to a computer implemented method and a scanner system. In particular, the disclosure relates to determining an inconsistent digital 3D scan and applying the inconsistent digital 3D scan to a current digital 3D scan.

### BACKGROUND

Intra oral scanning, i.e. scanning a patient's dentition in situ do obtain a digital dental impression, has become a common alternative to taking physical dental impressions. This has many advantages such as providing a better patient experience. The digital dental impression obtained by intra oral scanning can be processed digitally and e.g. restorative, orthodontic or other dental treatments can be simulated and designed digitally. In case a component for the treatment is required, e.g. a crown or an aligner, this can be manufactured based on a digital model by using 3D manufacturing machines, such as milling or 3D printing.

One of the important aspect of digital impression taking using an intra oral scanner (IOS) is to acquire the best possible digital impression during the patient visit. If the digital impression has errors, e.g. missing data or data representing unwanted components such as saliva, the subsequent treatment may fail and the patient has to be called in to the dentist to be scanned again, or worse, the treatment may not provide the desired result.

Accordingly, the IOS system may include different tools to correct or modify failed sections of the scan in order to obtain a useable scan. Alternatively, the scan probably has to be redone which prolongs the dentist visit.

In another situation the dentist may want to reuse a digital impression in a subsequent scan session and update the digital impression with data from a relevant area. This can for example be the case where

Scan and digital impression is used interchangeable where the scan represents the part of the dentition the dentist wanted to obtain. E.g. smaller intermediate scan, scan patches, only represent part of the full scan and is thus not a digital impression as should be understood herein.

One such tool is to mark areas that needs to be rescanned and then remove that area and replace it with new scan data. However, this introduces a manual step where the dentist or technician scanning needs to independently interact with the scanner system. Also, if the person scanner does not recognize an area as having incorrect data or the area was not properly marked errors will still be present in the final scan.

Accordingly, there still exists a need for correcting a part of scan during scanning of a patient in order to reduce the risk of obtaining final scans with errors. Also, the disclosure further relates to ensuring a continuous scan session without interruption where the dentist has to interact with the scanner interface.

### SUMMARY

An aspect of the present disclosure is to reduce the risk of obtaining final scans with errors.

A further aspect of the present disclosure is to ensure a continuous scan session without interruption where the dentist has to interact with the scanner interface.

According to the aspects, a computer implemented method for updating a current digital 3D scan representing the surface of a physical object with an at least one new 3D scan, where the updating of the current digital 3D scan provides an updated digital 3D scan representation of the surface of the physical object is disclosed. The computer implemented method may comprise obtaining the current digital 3D scan, obtaining the at least one new digital 3D scan, determining an inconsistent digital 3D scan by comparing the current digital 3D scan with the at least one new digital 3D scan, where at least a part of the new digital 3D scan does not overlap with at least a part of the current digital 3D scan, and creating the updated digital 3D scan representation, which represent the physical object by applying the inconsistent digital 3D scan to the current digital 3D scan.

The computer implemented method may comprise obtaining the current digital 3D scan which includes surface information of an area of the surface of the physical object Furthermore, the method includes obtaining the at least one new digital 3D scan that includes new surface information of at least a part of the area of the surface of the physical object. The obtaining of the at least one new digital 3D scan is done later than the obtaining of the current digital 3D scan. By for example, comparing the current digital 3D scan and the new digital 3D scan it is possible to identify any visual changes to the surface of the physical object, e.g. the tooth. Furthermore, the method may include determining an inconsistent digital 3D scan that includes new surface information of the physical object, and wherein the determining of the inconsistent digital 3D scan includes comparing the current digital 3D scan with at least one new digital 3D scan. The inconsistent digital 3D scan corresponds to differences in the surface information of the current digital 3D scan and of the at least one new digital 3D scan. The differences could be changes to the color of the surface, shape of the surface, progress in caries, progress in cracks, any visual changes of the surface or the physical object that affects the surface. At least a part of the new digital 3D scan does not partially overlap with at least a part of the current digital 3D scan, and the new digital 3D scan includes new information about an area of the surface of the physical object that corresponds to the non-partially overlap. A part of the new digital 3D scan and a part of the current digital 3D scan corresponds to an area of the surface of the physical object. The not partially overlap between the current digital 3D scan and the new digital 3D scan corresponds to an area of the surface where the surface information of both scans does not match, i.e. inconsistent digital 3D scan. Furthermore, the method includes determining the updated digital 3D scan representation which represent the physical object, by applying the inconsistent digital 3D scan to the current digital 3D scan such that surface information of the current digital is replaced with the new surface information.

The not overlap between the two scans do correspond to the same area of the surface of the physical object, and the not overlap scans means that the surface information of the two scans are different because of changes to the color of the surface, shape of the surface, progress in caries, progress in cracks, any visual changes of the surface or the physical object that affects the surface,

The computer implemented method may comprise obtaining the current digital 3D scan that includes surface information of an area of the surface of the physical object, obtaining the at least one new digital 3D scan that includes new surface information of at least a part of the area of the surface of the physical object, determining an inconsistent digital 3D scan, wherein the determining of the inconsistent digital 3D scan includes comparing the surface information with the new surface information, and where the inconsistent digital 3D scan includes at least a part of the new surface information that does not overlap with at least a part of the surface information, and determining the updated digital 3D scan representation which represent the physical object by applying the inconsistent digital 3D scan to the current digital 3D scan such that least part of the surface information of the current digital 3D scan is replaced with the at least part of the new surface information.

The disclosed method provides a method of scanning where inconsistent scan areas in a current digital scan are identified and automatically replaced with new digital 3D scans.

The at least one new digital 3D scan may be received from an intra oral scanner. The digital 3D scans obtained typically represents surfaces, such as the surfaces of a dental object, such as a restoration, a tooth or teeth, the upper and/or lower jaw of a patient etc.

According to the aspects, a scanner system for intraoral scanning of a dental object is disclosed. The scanner system may comprise a scanning probe for receiving images of the dental object, a peripheral output device for visualising a digital 3D representation of the dental object and a computer processor coupled to the scanning probe and the peripheral output device. The computer processor may receive data from the scanning probe and outputs computed data to the peripheral output device, wherein the computer processor comprises a method for updating a current digital 3D scan representing the surface of the dental object with an at least one new 3D scan to provide an updated digital 3D scan representation of the surface of the dental object as disclosed herein.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates the steps of one embodiment of a computer implement method disclosed herein,

FIGS. 2a, 2b and 2c, illustrate outcomes discussed in relation to FIG. 1 above,

FIG. 3 illustrates the steps of another embodiment of a computer implemented method disclosed herein, and

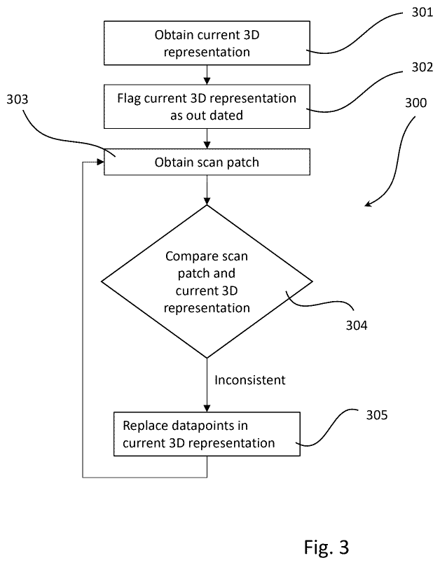

FIG. 4 illustrates an embodiment of a scanner system as disclosed herein.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

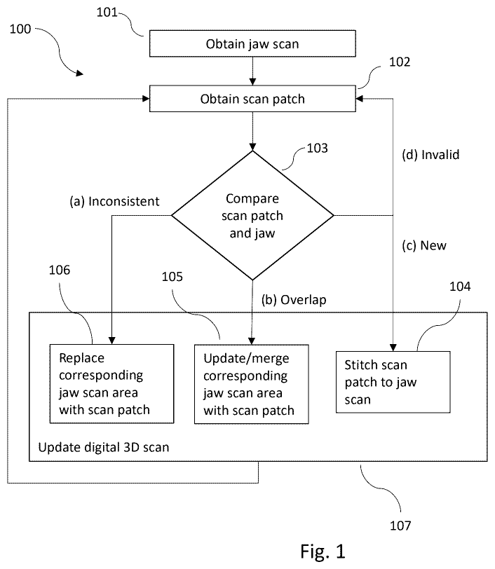

FIG. 1 is schematically illustrating an exemplary of a computer implemented method for updating a current digital 3D scan representing the surface of a physical object 100. The physical object is a dental object of a patient and the method described with respect to FIG. 1 is where the scanning process relates to the same dental object of a patient.

In step 101 a jaw scan is obtained, i.e., a scan of the jaw(s) of a patient. The jaw scan is not necessarily complete and can thus be a partly jaw scan. The jaw scan can be obtained directly, e.g. through intra oral scanning, or it can be loaded into the computer from an earlier scanning session, for example, from scanning a physical impression of the patient dentition.

Alternatively, instead of the jaw scan other types of current digital 3D scans may be obtained. For example, the current digital 3D scan can for example represent at least a part of an oral cavity of a patient, a jaw in the oral cavity of a patient, a preparation, a tooth of an oral cavity of a patient and/or a restoration. Accordingly, these terms are not limiting and can be used interchangeably throughout the disclosure.

A scan patch is subsequently obtained in step 102. The scan patch is a small digital representation of section of the jaw of the patient. Similarly to the jaw scan above other type new digital 3D scans, such as a scan patch, may be obtained.

Typically, the current digital 3D scan and the new digital 3D scan may represent the same dental object, however, in some cases it may be different. E.g. the current digital 3D scan may represent a dental preparation where the new digital 3D scan may represent a crown restoration (or a part thereof) arranged on the dental preparation.

In step 103, the scan patch and the jaw scan are compared in order to determine one of the following outcomes,
(a) that the scan patch is an inconsistent scan surface of the jaw scan, where at least a part of the scan patch does not overlap with at least a part of the jaw scan.
(b) that the scan patch is an overlapping scan surface of the jaw scan, or
(c) that the scan patch is a new scan surface of the jaw scan.

The outcome (a) that the scan patch is an inconsistent scan surface of the jaw scan occurs when the scan patch corresponds to a part of the jaw scan but is inconsistent since it does not fulfil certain criterion.

In one embodiment as shown in step 103 an inconsistency criterion may be established which defines when the new digital 3D scan does not overlap the current digital 3D scan. When comparing the scan patch and the jaw scan the method checks the scan patch against the inconsistency criterion and determines the scan patch as inconsistent if it complies with the inconsistency criterion. A part of the new digital 3D scan not overlapping the current digital 3D scan implies that the part of the new digital 3D scan does not match with the current digital 3D scan.

Thus, it may be understood that in other embodiments it is disclosed that the step of determining an inconsistent digital 3D scan may further comprise:
establishing an inconsistency criterion which defines when the new digital 3D scan does not overlap the current digital 3D scan,
checking the new digital 3D scan against the inconsistency criterion, and
determining the inconsistent digital 3D scan as the part of the at least one new digital scan that comply with the inconsistency criterion.

The inconsistency criterion can for example in one embodiment be a distance threshold limit or a distance threshold interval, wherein the new digital 3D scan, e.g. scan patch, comply with the inconsistency criterion when the distance between the at least new digital 3D scan, e.g. scan patch, and the current digital 3D scan, jaw scan, is in the distance threshold limit or interval. Typically, comparing distances will be done by looking at distances between data points in the two scans. A matching algorithm can for example be used to determine corresponding data points between the two scan, and if the average distance between corresponding data points are above the distance threshold the scan patch is inconsistent.

In this context a distance may be understood as a distance in space e.g., morphology or cartesian distance or a Euclidean distance in color space such as RGB or LAB colors. Classification based on semantic segmentation like tooth or gingiva, surface properties like roughness or scattering properties. In other words, a distance should be understood as a difference or discrepancy in one or more characteristics on data point level.

A data point may be understood as one 3D point in a point cloud, an ensemble of 3D points, such as a group, or neighbours and/or next-neighbour points. A vertex in a mesh, or a collection of vertices in a mesh, one or more voxels, likelihood distributions, color information in RGB or LAB color space, surface normals or any appropriate collection of individual data points in the digital 3D scan.

In one embodiment the step 103 of comparing the scan patch with the jaw scan to determine an inconsistent digital 3D scan may involve identifying the area of the jaw scan that corresponds to the scan patch.

In one embodiment as disclosed in FIG. 1, this step may further comprise projecting the inconsistent digital 3D scan (scan patch) to the current digital 3D scan (jaw scan) in step 103, and applying the data of the inconsistent 3D scan (scan patch) to the corresponding data of the projected area on the current digital 3D scan (jaw scan) in step 106.

The projection may be done in a number of different ways.

In one embodiment the inconsistent digital 3D scan (scan patch) is projected to the current digital 3D (jaw scan) scan along a line of sight axis.

In case an intra oral scanner has been used to obtain the scan patch the line of sight axis may correspond to the optical axis of an intra oral scanner used to obtain the at least one new digital 3D scan (scan patch).

In another embodiment the individual data points in the new digital 3D scan (scan patch) are analysed when establishing inconsistency by evaluating the inconsistency criteria applied to the current digital 3D scan.

Other means of determining corresponding surfaces can be done by for example other projecting methods. In some embodiments corresponding surfaces may be found by e.g. determine closest points between the scan patch and the jaw scan, for example by using a matching algorithm. In other embodiment the computer implement method may look along the surface normal of the scan and use these for projecting, apply feature or landmark detection or any other suitable method to obtain correspondence between data points of the inconsistent digital 3D scan and the current digital 3D scan.

As discussed herein identifying an inconsistent digital 3D scan can be done in many different ways and a person skilled in the art presented by the current disclosure will be able to provide further embodiments understanding that inconsistent digital 3D scan(s) may be determined when looking at two scans, typically comparing these, and looking for surfaces or data that do not overlap but represent new information about an area that has already been obtained.

For example a tooth where the morphology has changed will not create overlapping scans, but a new scan of surface that replaces a previous surface. The method as disclosed determines this by way of looking at inconsistencies between the two surfaces and may for example determine that the new surface should replace the previous surface.

In some embodiments corresponding scan areas are identified to establish what should be compared by using e.g. the inconsistency criterion.

For example, determining corresponding scan areas may be relevant when the inconsistent digital 3D scan represent inconsistent scan areas or inconsistent scan sections of a larger scan. It can for example be an inconsistent scan patch as discussed, where the scan patch represent a part or a section of a larger scan.

In general the inconsistency criterion can be understood as setting the rules for determining when corresponding 3D scans are inconsistent. E.g. when corresponding data point do not fall within the criterion, or within an interval of the criterion.

The outcome (b) that the scan patch is an overlapping scan surface of the jaw scan occurs by determining an overlapping digital 3D scan, where the new digital 3D scan (scan patch) overlaps with at least a part of the current digital 3D scan (jaw scan).

In the current embodiment the full scan patch (e.g. new digital 3D scan) overlaps in order to be determined as overlapping, however, in another embodiment the scan patch may be considered as overlapping when only a part of the scan patch overlaps.

Similarly, to the inconsistency criterion the step of determining an overlapping digital 3D scan may in one embodiment comprise establishing an overlap criterion, which defines when the new digital 3D scan, scan patch, overlap the current digital 3D scan, jaw scan. Accordingly, the new digital 3D scan is checked against the overlap criterion and is determining the new digital 3D scan as an overlapping digital 3D scan if the at least one new digital 3D scan that comply with the overlap criterion.

The overlap criterion may comprise a topology threshold, wherein the new digital 3D scan comply with the overlap criterion when the match between the surface of the new digital 3D scan and at least a part of the current digital 3D scan is above the topology threshold.

In one embodiment the overlap criterion may be that the new digital 3D scan, jaw scan, does not comply with the inconsistency criterion. E.g. if the distances between data points are below a distance threshold set out in the inconsistency criterion then the scan patch overlap with the jaw scan.

Determining that (c) the scan patch is a new scan surface of the jaw scan may in one embodiment occur when outcomes (a) that the scan patch is inconsistent and (b) that the scan patch is overlapping cannot be determined but the scan patch can be stitched to the jaw scans.

An invalid scan patch may in one embodiment be determined (d) if none of the outcomes (a), (b) and (c) can be established. In this case the invalid scan patch is discarded or disregarded in step 103 and the scanning process returns to step 102 where a new scan patch may be obtained.

If the comparing step 103 provides any of the outcomes (a) inconsistent scan patch, (b) overlapping scan patch and (d) new scan patch the method may subsequently update the jaw scan in an updating step 107.

If the scan patch is identified as an inconsistent scan surface as outcome (a) an updated digital 3D scan, i.e. an updated jaw scan in the current embodiment, is created in updating step 106. The updated jaw scan which represent the physical object is created by applying the inconsistent digital 3D scan, i.e. scan patch, to the current digital 3D scan, i.e. jaw scan. Step 106 has also for some embodiments been described together with outcome (a) in step 103 above as these steps are practically often performed together and thus reading is facilitated by keeping it together in the current disclosure.

In one embodiment applying the inconsistent digital 3D scan (scan patch) to the current digital 3D scan may comprise overwriting the data of the projected area on the current digital 3D scan (jaw scan) with the data of the corresponding projected inconsistent digital 3D scan (scan patch).

Alternatively, applying the inconsistent digital 3D scan (scan patch) to the current digital 3D scan may comprise adding the data of the inconsistent digital 3D scan to the corresponding data in the projected area on the current digital 3D scan (jaw scan) in a weighted manner depending on the distance value of each corresponding data point. Such that, if the distance value of corresponding datapoints are high, the weight of the added data point from the inconsistent digital 3D scan is high relative to the corresponding data point on the current digital 3D scan.

This will in other words have the effect that the new data point of the inconsistent scan patch (inconsistent digital 3D scan) will pull the surface of the jaw scan (current digital 3D scan) to its location. This creates a smooth transition between new inconsistent data and the current data along the boundaries that suppresses noise and blend both surface and/or color.

The individual data points in the new digital 3D scan may alternatively or additionally be applied to the current digital 3D scan in a weighted order dependent on distance values or in a discrete manner.

In one example, such as a weighted order, data points of the new digital 3D scan are applied the current digital 3D scan relative to the distance value between the corresponding data points.

In another example, such as a divided order, data points of the new digital 3D scan with a distance measure above a threshold value relative to corresponding data points in the current digital 3D scan are applied in a first way and data points of the new digital 3D scan with a distance measure below a threshold value relative to corresponding data points in the current digital 3D scan are applied in second way.

The first and second way may be either a discrete order or continuous weighted order or a combination.

If a scan patch is identified as an overlapping digital 3D scan as outcome (b) in step 103 then the updating step 107 may comprise the step 105 of merging the overlapping digital 3D scan (scan patch) with the current digital 3D scan (jaw scan) in order to obtain the updated digital 3D scan (updated jaw scan).

If a scan patch is identified as a new scan surface of the jaw scan as outcome (c) in step 103 then the updating step 107 may comprise the step 104 of stitching the scan patch to the jaw scan.

Generally speaking, this would be the typically scanning operation of an intra oral scanner where scan patches are added to the jaw scan thereby expanding the surface/volume of the scan. Stitching scans are generally known and involves identifying common references in the scan scans to be stitched and then combine the scans by aligning them at the common references.

As mentioned the scanning may be done by an intra oral scanner. In such a case multiple new digital 3D scans (scan patches) are received continuously as a scan stream. Accordingly the scan patches needs to be continuously compared to the jaw scan in step 103. This occurs for example in the embodiment of FIG. 1 where the method returns to receiving the next scan patch in the scan stream in step 102 after the jaw scan was updated with a previous scan patch in step 107.

Thus, as an example the method may comprise steps that continuously stitch the multiple new digital 3D scans (scan patches) to each other.

Similarly, the method may comprise steps that continuously determining the inconsistent digital 3D scan, where at least a part of the at least one new digital 3D scan does not overlap with at least a part of the current digital 3D scan, and continuously creating the updated digital 3D scan, which represent the physical object by applying the inconsistent digital 3D scan to the current digital 3D scan.

Additionally, the computer implemented method may comprise steps of continuously checking the at least one new digital 3D scan against the inconsistency criterion, and/or continuously determining an overlapping digital 3D scan where at least a part of the new digital 3D scan overlaps with at least a part of the current digital 3D scan.

It should be understood that when determining an inconsistent digital 3D scan or an overlapping digital 3D scan that these are not necessarily established in the computer implement method as independent scan objects or files. In one embodiment annotations can be done in the current digital 3D scan or the new digital 3D scan identifying part therein as inconsistent digital 3D scan and/or overlapping digital 3D scan.

Also, when creating the updated digital 3D scan blending algorithms may be used when the inconsistent digital 3D scan or the overlapping digital 3D scan are applied to the current digital 3D scan. This avoids jagged edges/overlaps, unnatural changes in color etc. Such blending algorithms or techniques are known and used by persons skilled in the art when combining 3D digital models.

The scans obtained may be different types of scan, e.g. they may store data and represent the scanned object in different ways. For example, the digital 3D scans may be volumetric information, meshes or point clouds.

Furthermore, it should be understood that in some embodiments the scans represent the geometry of the surface of an object. However, additional information may also be obtained and updated in the method disclosed herein. Such information can for example be color, fluorescence and infrared.

For example the inconsistency criterion and/or the overlap criterion may also include requirements to such additional information.

While the scanning the jaw as discussed in FIG. 1 and as disclosed herein the scanning method may further include the step of pre-processing the at least one new digital 3D scan (scan patch).

One pre-processing step can for example be a so-called soft tissue removal process where the pre-processing applies a removing process, for removing unwanted scan elements, to the at least one new digital 3D scan. Soft tissue can for example be cheeks or the tongue that gets in the way while scanning. The pre-processing can also remove metal objects such as a dentist mirror or other dentist tools.

The removal process can for example be a trained machine learning algorithm.

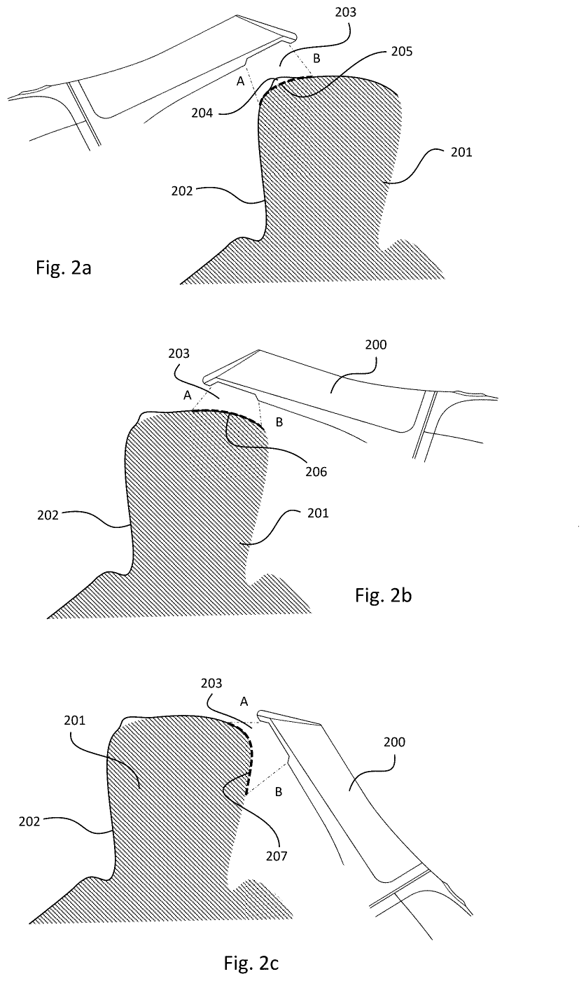

FIGS. 2a, 2b and 2c illustrate by examples different practical scanning situations that may result in the different scanning outcomes (a), (b) and (c) as described above.

FIG. 2a shows an intra oral scanner 200 scanning a tooth 201 in a jaw. The tooth is represented by the hatched area. A current digital scan 202, e.g. tooth scan, of at least a part of the tooth has already been obtained and is shown as a solid line enclosing the hatched area representing the tooth.

The intra oral scanner obtains images subsections of the tooth while scanning. A subsection is determined in part by the field of view 203 defined by the broken lines A and B. The images taken by the subsections can subsequently be further processed to a scan patch, which is a partial 3D representation of the object scanned by the intra oral scanner.

In FIG. 2a an inconsistent section 204 has been recorded during a previous scan section. As illustrated it can be seen that in the inconsistent section the current digital scan does not follow the hatched area representing the tooth. Such an inconsistent section can for example have been created previously if saliva or a foreign object have been present during that scanning section. However, in the current scan session the intra oral scanner obtains a scan patch 205, shown by the hatched line segment, where the surface represented in the scan patch follows the surface of the tooth.

The method as disclosed herein will in one embodiment as discussed determine (a) that the scan patch is an inconsistent scan surface of the tooth scan 202, where at least a part of the scan patch does not overlap with at least a part of the jaw scan 202.

An updated tooth scan (not shown) may thus be created by overwriting the data of the tooth scan 202 in the field of view 203, with the data in the scan patch that was taken in the field of view 203.

As discussed herein the inconsistent digital 3D scan, e.g. the scan patch 204 as discussed with respect to FIG. 2a, is not necessarily a result of an error in scanning. It may also be used to update a scan of a tooth or other dental object after it has been modified. For example, if it is planned to provide a crown on a tooth three scanning steps may be performed, the first on the original tooth which may be damaged and a crown treatment is hence planned. The tooth is subsequently prepped and a second scanning session is performed where inconsistent scans are determined where the tooth has been prepped and the first scan is updated with the inconsistent scans so that it represents the prepped tooth. Finally, after the crown has been placed a third scanning session may be performed where inconsistent scans are determined based on the updated scan representing the prepped tooth and the scans of the crown received in the third scanning session. Accordingly, a further update of the scan can be done where the part of the scan representing the prepped tooth is overwritten with the scan representing the crown.

FIG. 2b illustrates an embodiment where the method determined (b) that the scan patch 206, shown by the hatched line segment superposed onto the line segment representing the jaw scan 202, is an overlapping scan surface of the tooth scan and merges the scan data of the scan patch with the tooth scan in the field of view 203 of the intra oral scanner.

In another situation as shown in FIG. 2c the method determines (c) that the scan patch 207, represented by the hatched line segment, is a new scan surface of the jaw scan. This is a situation where the method determined that the scan patch has some overlap with the tooth scan 202 but surface has been obtained that has no reference, e.g. it cannot be determine as overlapping nor inconsistent. In such situation the method in the current embodiment determines that the scan patch represent a new surface of the tooth 201 and stiches it to the tooth scan 202, effectively increasing the obtained area of the scanned surface of the tooth.

Another embodiment 300 of a computer implemented method for updating a current digital 3D scan representing the surface of a physical object with an at least one new 3D scan is shown in FIG. 2.

In step 301 a sequence of scan patches are obtained which are used to generate a current digital 3D representation. The scan patches which each represents portions of the dental object captured are via a dental imaging device, such as an intra oral scanner.

In step 302 the current digital 3D representation is flagged as “out dated”. This can be done manually by a user input, it can done if the current digital 3D representation is loaded into the system and/or has a time stamp older than a predetermined time. As can be understood by the person skilled in the art, many other criterion for flagging the current 3D representation as outdated can be established.

In step 303 a subsequent scan patch is obtained.

In step 304 the subsequent scan patch is compared to the current digital 3D model to determine if the at least one subsequent scan patch is inconsistent with the current digital 3D model.

This may be achieved by determining if datapoints in at least part of the subsequent scan patch is different from at least a part of the current digital 3D representation.

If it is determined that the scan patch is inconsistent an updated digital 3D representation may be generated by replacing datapoints from the inconsistent scan patch with the corresponding datapoints in the current digital 3D representation in step 305.

After updating the current digital 3D representation in step 305 the method may return to step 303 and obtain yet a further subsequent scan patch.

In some embodiments a step of aligning the new 3D scan to the current digital 3D scan. This is typically in case where the current digital 3D scan has been taken in a different scan session and the new 3D scan needs to be arranged in the same coordinate system as the current digital 3D scan.

Accordingly it should be understood that overlapping and inconsistent scan surfaces as discussed herein can be both aligned and non-aligned. In general as discussed herein alignment relates to bringing data sets or surface representations, e.g. the current digital 3D scan such as the jaw scan and the at least one new digital 3D scan such as the scan patch, into a common coordinate system. Thus, as for example discussed in FIG. 1, inconsistent scan patches in outcome (a) may be aligned when stitched together with previous scan patches, overlapping scan patches in outcome (b) may be aligned when stitched together with previous scan patches and new scan patches in outcome (c) may be aligned when stitched together with previous scan patches.

However, in one embodiment aligning a new 3D scan to the current digital 3D scan may be achieved by using an overlapping digital 3D scan.

In other embodiment the step of aligning the at least one new 3D scan to the current digital 3D scan comprises using alignment marker data from physical markers at the physical object.

The updated digital 3D scan is typically output to a peripheral output device, such as a monitor, where a user may follow visually while scanning a patient. The monitor may for example be part of a scanning system, such as an intra oral scanner.

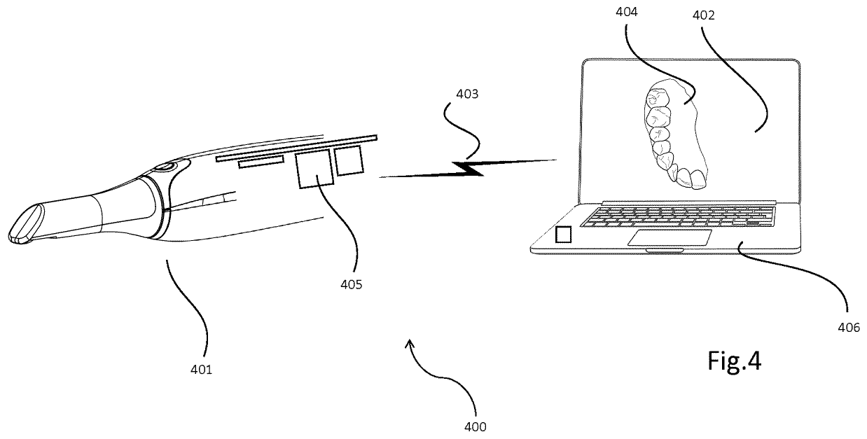

An embodiment of a scanner system 400 for intra oral scanning of the dental object is shown in FIG. 4.

The scanner system may comprise a scanning probe 401 for receiving images of the dental object.

A laptop computer 406, having a peripheral output device 402 in the shape of a monitor for visualising the digital 3D representation 404 of the dental object can be provided in wireless communication with the scanning probe. Alternative a desktop computer or other computing device, e.g. table, can be provided in communication with the scanning probe. The communication may be done wirelessly as described or wired.

A computer processor 405 and other electronic hardware may be arranged in the scanning probe and enables the wireless communication via a controller 406 in the scanning probe and a controller 407 in the lap top computer 406.

The laptop receives data from the scanning probe and outputs computed data to the monitor.

The computer implemented method for updating a current digital 3D scan representing the surface of the dental object with an at least one new 3D scan to provide an updated digital 3D scan representation of the surface of the dental object as discussed herein can be performed by the scanning system 400.

The computer processor provided in the scanning probe and in the laptop computer is typically made up of different types of electronic hardware. The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

The scanner system disclosed herein may be an intraoral scanning device such as the TRIOS series scanners from 3Shape A/S or a laboratory-based scanner such as the E-series scanners from 3Shape A/S. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled during a scanning session, such that at least some sets of sub-scans overlap at least partially, in order to enable stitching during scanning. The result of stitching is the digital 3D representation of a surface larger than that which can be captured by a single sub-scan, i.e. which is larger than the field of view of the 3D scanning device. Stitching, also known as registration, works by identifying overlapping regions of 3D surface in previous sub-scans/recorded 3D surface and transforming the new sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The dental scanning system preferably further comprises a processor configured to generate scan data (intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infrared (IR) images, fluorescence images, reflectance color images, x-ray images, and/or combinations thereof.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to an component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. A computer implemented method for updating a current digital 3D scan representing the surface of a physical object with an at least one new 3D scan, where the updating of the current digital 3D scan provides an updated digital 3D scan representation of the surface of the physical object,
wherein the computer implemented method comprises the steps of:
obtaining the current digital 3D scan that includes surface information of an area of the surface of the physical object,
obtaining the at least one new digital 3D scan that includes new surface information of at least a part of the area of the surface of the physical object,
determining differences in the surface information of the current digital 3D scan and of the at least one new digital 3D scan, wherein the determining of the differences includes comparing the surface information with the new surface information, and where the differences includes at least a part of the new surface information that does not overlap with at least a part of the surface information, and
determining the updated digital 3D scan representation which represent the physical object by applying the differences to the current digital 3D scan such that the at least part of the surface information of the current digital 3D scan is replaced with the at least part of the new surface information, and wherein the differences includes one or more of following:
change in color of the surface of the object,
change in shape of the surface of the object,
progress in caries, and
progress in cracks.

2. A computer implement method according to claim 1, wherein the step of determining the inconsistent digital 3D scan further comprises:
establishing an inconsistency criterion which defines when the new digital 3D scan does not overlap the current digital 3D scan,
checking the new digital 3D scan against the inconsistency criterion, and
determining the differences in the surface information of the current digital 3D scan and of the at least one new digital 3D scan as the part of the at least one new digital scan that comply with the inconsistency criterion.

3. A computer implemented method according to claim 2, wherein the inconsistency criterion comprises a distance threshold, wherein the new digital 3D scan comply with the inconsistency criterion when the distance between the at least new digital 3D scan and the current digital 3D scan is above the distance threshold.

4. A computer implemented method according to claim 1, wherein the method further comprises the step of:
determining an overlapping digital 3D scan, where at least a part of the new digital 3D scan overlaps with at least a part of the current digital 3D scan.

5. A computer implements method according to claim 4, wherein the step of creating the updated digital 3D scan further comprises merging the overlapping digital 3D scan with the current digital 3D scan.

6. A computer implemented method according to claim 4 wherein the step of determining an overlapping digital 3D scan further comprises,
establishing an overlap criterion which defines when the new digital 3D scan overlap the current digital 3D scan,
checking the new digital 3D scan against the overlap criterion, and
determining the overlapping digital 3D scan as the part of the at least one new digital 3D scan that comply with the overlap criterion.

7. The computer implemented method according to claim 1, where the method further comprises the step of aligning the new 3D scan to the current digital 3D scan.

8. The computer implemented method according to claim 7, wherein the step of aligning the new 3D scan to the current digital 3D scan comprises using the overlapping digital 3D scan.

9. The computer implemented method according to claim 7, wherein step of aligning the at least one new 3D scan to the current digital 3D scan comprises using alignment marker data from physical markers at the physical object.

10. The computer implement method according to claim 1, wherein the at least one new digital 3D scan is received from an intra oral scanner.

11. The computer implemented method according to claim 1, wherein multiple new digital 3D scans are received continuously as a scan stream.

12. A computer implemented method according to claim 12, wherein the method further comprises the step of,
continuously stitching the multiple new digital 3D scans to each other.

13. The computer implemented method according to claim 1, wherein at least the below steps of the computer implemented method is performed continuously,
determining differences in the surface information of the current digital 3D scan and of the at least one new digital 3D scan, wherein the determining of the differences includes comparing the surface information with the new surface information, and where the inconsistent digital 3D scan includes at least a part of the new surface information that does not overlap with at least a part of the surface information, and
determining the updated digital 3D scan representation which represent the physical object by applying the differences to the current digital 3D scan such that the at least part of the surface information of the current digital 3D scan is replaced with the at least part of the new surface information, and wherein the differences includes one or more of following:
change in color of the surface of the object,
change in shape of the surface of the object,
progress in caries, and
progress in cracks.

14. The computer implemented method according to claim 13, wherein any one of the following steps of the method are performed continuously,
checking the at least one new digital 3D scan against the inconsistency criterion, and/or
determining an overlapping digital 3D scan where at least a part of the new digital 3D scan overlaps with at least a part of the current digital 3D scan.

15. A computer implemented method according to claim 1, wherein the step of creating the updated digital 3D scan, which represent the physical object by applying the differences in the surface information of the current digital 3D scan and of the at least one new digital 3D scan to the current digital 3D scan further comprises,
projecting the differences to the current digital 3D scan, and
applying the data of the differences to the data of the projected area on the current digital 3D scan such that the at least part of the surface information of the current digital 3D scan is replaced with the at least part of the new surface information.
